(19) United States
(12) Patent Application Publication
(10) Pub. No.: US 2005/0042193 A1
(43) Pub. Date: Feb. 24, 2005

(54) Graft copolymers in cosmetic formulations

(76) Inventors: Klemens Mathauer, Heidelberg (DE); Wolfgang Jahnel, Bellheim (DE); Peter Hossel, Schifferstadt (DE)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/503,367

(22) PCT Filed: Feb. 4, 2003
(86) PCT No.: PCT/EP03/01064

(30) Foreign Application Priority Data
Feb. 15, 2002 (DE) 10206596.9

(51) Int. Cl. A61K 7/06; A61K 7/11

(52) U.S. Cl. 424/70.16; 525/65

(57) ABSTRACT

Disclosed are graft copolymers which are obtained by means of radical graft copolymerization of a) N-vinylformamide, b) one or several copolymerizable monomers of general formula (I), in which n=0.1 and R¹ and R² independently represent H, C₁-C₄ alkyl, CN, COOH, and/or one or several unsaturated sulphonic acids and/or one or several unsaturated phosphonic acids on a polymeric graft base c) of general formula (II), in which R═H, —CH₃, and n=5-50 000.

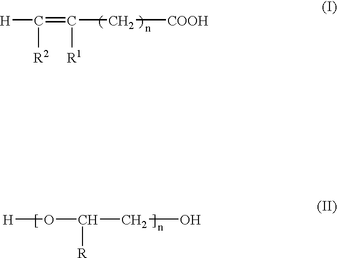

The present invention relates to the use of graft copolymers as a component in cosmetic compositions. The graft copolymers are formed by grafting monoethylenically unsaturated open-chain monomers containing N-vinylamide units onto a polymeric grafting base.

Polymers are widely used in hair cosmetics. Their object in hair cosmetics is to influence the properties of the hair, in particular to set the hair, to improve the combability and to impart a pleasant feel.

Thus, conditioners are used for improving the dry and wet combability, feel, gloss and appearance and for imparting antistatic properties to the hair. Water-soluble polymers having polar, frequently cationic functionalities, which have a relatively great affinity to the structurally related negative surface of the hair, are preferably used. The structure and mode of action of various hair treatment polymers are described in Cosmetics & Toiletries 103 (1988), 23. Commercial conditioner polymers are, for example, cationic hydroxyethylcellulose, cationic polymers based on N-vinylpyrrolidone, for example copolymers of N-vinylpyrrolidone and quaternized N-vinylimidazole, acrylamide and diallyldimethylammonium chloride or silicones.

For setting hairstyles, vinyllactam homo- and copolymers and carboxylate-containing polymers are used. Requirements for hair setting resins are, for example, strong setting at high atmospheric humidity, elasticity, capability of being washed out from the hair, tolerance in the formulation and a pleasant feel of the hair.

Difficulties are often presented by the combination of different properties, for example strong setting, pleasant feel of the hair and simultaneous thickening effect of the polymers in the cosmetic hair formulations.

This is particularly important in gel formulations. Moreover, conventional hair setting polymers frequently have incompatibilities with thickener polymers, resulting in turbidity and precipitation in the cosmetic formulations. Classical thickeners, which consist either of crosslinked polyacrylic acid (Carbopol) or of copolymers, have the disadvantage that, owing to the crosslinking, they do not form films suitable for hair setting. They ensure the consistency of the gel but, after drying of the gel on the hair, are no longer required and therefore potentially adversely affect the performance characteristics of the formulation (setting effect, moisture sensitivity).

WO-A-96/03969 describes haircare compositions comprising an N-vinylformamide homopolymer or a copolymer of N-vinylformamide units and a further vinyl monomer selected from styrenes, alkyl esters of acrylic and methacrylic acid, vinyl esters of the formula CH₂═CH—OCO-alkyl, N-alkyl-substituted acrylamides and methacrylamides, esters of fumaric, itaconic and maleic acid, vinyl ethers, hydroxy-functionalized acrylates and methacrylates, acrylamide, non-alkyl-substituted acrylamides and cyclic amides. The specific example of a cyclic amide is N-vinylpyrrolidone. Further examples of vinyl monomers are secondary, tertiary and quaternary amines, such as dimethyldiallylammonium chloride, dimethylaminoethyl methacrylate or dimethylaminopropyl methacrylate.

DE 19640363 describes copolymers of N-vinylformamide and quaternized N-vinylimidazole and the uses thereof in cosmetics.

DE 19907587.5 describes the use of polymers which are obtainable by free radical polymerization of at least one vinyl ester in the presence of polyether-containing compounds and, if appropriate, of one or more copolymerizable monomers and subsequent at least partial hydrolysis of the ester function in cosmetic hair formulations. Inter alia, vinylformamide is mentioned as an example of copolymerizable monomers.

DE-A1-44 09 903 describes graft polymers containing N-vinyl units, processes for their preparation and their use. Monoethylenically unsaturated monomers are grafted on a grafting base which is a polymer which contains in each case at least 5% by weight of units of the formulae 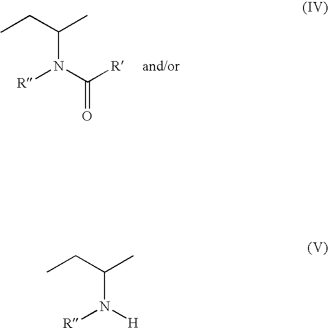
where R′ and R″ are H or C₁- to C₆-alkyl. Suitable monoethylenically unsaturated monomers are any ethylenically unsaturated monomers whose polymerization is not inhibited by the amino groups in free or in salt form, for example monoethylenically unsaturated mono- and dicarboxylic acids, and the salts and esters thereof with C₁- to C₃₀-alcohols. Suitability of these graft copolymers as an active ingredient in cosmetic formulations is not mentioned.

WO 96/34903 describes graft polymers containing N-vinyl units, processes for their preparation and their use. Monoethylenically unsaturated monomers are grafted on a grafting base which is a polymer which contains at least 3 units of a C₂- to C₄-alkylene oxide and/or contains polytetrahydrofuran, and then at least partial hydrolysis is effected. Suitability of these graft copolymers as an active ingredient in cosmetic formulations is not mentioned. Graft polymers according to claim 1 are not described.

U.S. Pat. No. 5,334,287 discloses graft polymers-which are obtainable by free-radically initiated polymerization of N-vinylcarboxamides, preferably N-vinylformamide, and, if appropriate, other monomers in the presence of monosaccharides, oligosaccharides, polysaccharides or the respective derivatives thereof and, if appropriate, hydrolysis of the N-vinylcarboxamide group incorporated in the form of polymerized units, with formation of vinylamine units. Suitability of these graft copolymers as an active ingredient in cosmetic formulations is not mentioned.

In WO 9825981, amphiphilic graft polymers are synthesized by grafting hydrophobic monomers, e.g. styrene, onto polymers which contain structural elements of the formula (IV) and/or (V). The graft polymers obtained are used, inter alia, as additives for cosmetic formulations.

In DE-A1-196 40 363, the use of water-soluble copolymers as an active ingredient in cosmetic formulations is claimed. The copolymer contains, as a characteristic structural element, units of the formula (VI) 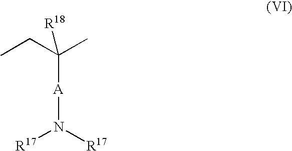
where A is a chemical bond or an alkylene group, the radicals R¹⁷, independently of one another, are H, alkyl, cycloalkyl, aryl or aralkyl and R¹⁸ is H, alkyl or aralkyl.

Personal care creams which contain a monoaldehyde-modified vinylamine polymer are disclosed in U.S. Pat. No. 5,270,379.

Copolymers which are used, for example, as hair setting compositions are composed of N-vinylamide monomers of the formula 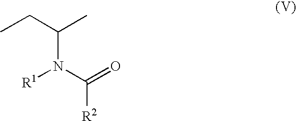
where R¹ and R² are H or C₁-C₅-alkyl, and the comonomer is selected from vinyl ethers, vinyllactams, vinyl halides, vinyl esters of monobasic saturated carboxylic acids, (meth)acrylic esters, (meth)acrylamides and (meth)acrylonitriles and esters, anhydrides and imides of maleic acid, are disclosed in DE 14 95 692.

U.S. Pat. No. 4,713,236 describes hair conditioners based on polymers containing vinylamine units. In particular, polyvinylamine and its salts, α-substituted polyvinylamines, e.g. poly(α-aminoacrylic acid) or copolymers which, in addition to vinylamine, contain comonomers such as vinyl alcohol, acrylic acid, acrylamide, maleic anhydride, vinyl sulfonate and 2-acrylamido-2-methylpropanesulfonic acid incorporated in the form of polymerized units are mentioned.

It is an object of the present invention to provide polymers which are very useful for cosmetic applications and, for example in the area of hair cosmetics, have good performance characteristics, such as a pleasant feel, and at the same time a good conditioning effect or good setting effect and simultaneously a thickening property. In this context, the solubility of the polymers in conventional cosmetic hair formulations to give a clear solution was of particular interest.

It is an object of the present invention to provide polymers which meet both requirements for hair cosmetics (setting effect, curl retention) and performance requirements (thickening properties, solubility to give a clear solution).

It is an object of the present invention to provide copolymers which combine a thickening effect (after neutralization of the polymer with bases customary for cosmetics) with film-forming properties, so that these polymers can be used, for example, as setting polymers in hair gel formulations.

There was particular interest in polymers which have thickening properties even without the addition of further, conventional thickeners (for example high molecular weight polyacrylic acids of the Carbopole® type).

We have found that this object is achieved, according to the invention, by graft copolymers which are obtainable by free radical graft copolymerization of
a) N-vinylformamide,
b) one or more copolymerizable monomers of the formula (I) 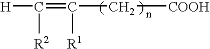
   where n is 0 or 1 and R¹ and R², independently of one another, are H, C₁-C₄-alkyl, CN or COOH,
and/or one or more unsaturated sulfonic acids
and/or one or more unsaturated phosphonic acids
onto a polymeric grafting base c) of the formula

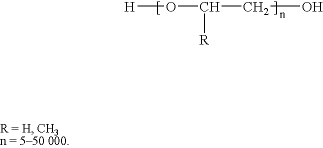

In the preparation of the polymers used according to the invention, grafting onto the polymeric grafting base c) may occur during the polymerization, which may lead to the advantageous properties of the polymers. However, other grafting mechanisms are also conceivable.

Depending on the degree of grafting, the polymers used according to the invention are to be understood as meaning both pure graft polymers and mixtures of the abovementioned graft polymers with ungrafted compounds c) and homo- or copolymers of the monomers (a) and (b).

The novel polymers are preferably water-soluble or water-dispersible.

Here, water-soluble polymers are to be understood as meaning polymers which have a solubility of at least 1 g/l at 20° C. in water. Here, water-dispersible polymers are to be understood as meaning polymers which disintegrate into dispersible particles with stirring.

For the preparation of the novel polymers, N-vinylformamide is used as monomer a).

One or more copolymerizable monomers of the formula (I) 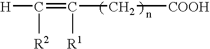
where n is 0 or 1 and R¹ and R², independently of one another, are H, C₁-C₄-alkyl, CN or COOH,
and/or one or more unsaturated sulfonic acids and/or one or more unsaturated phosphonic acids are used as monomer b).

Monomers of the formula (I) are particularly preferred.

Suitable C₁-C₄-alkyl radicals are, for example, methyl, ethyl, n-propyl, isopropyl, n-butyl and tert-butyl.

Examples of monomers of the formula (I) are acrylic acid, methacrylic acid, maleic acid, fumaric acid, crotonic acid, isocrotonic acid, vinylacetic acid and itaconic acid.

Monomers of the formula (I) where n is 0 are particularly preferred. Acrylic acid and methacrylic acid are particularly preferred.

Suitable unsaturated sulfonic acids are all compounds of the formula R—SO₃H, where R is a radical having at least one ethylenically unsaturated group.

Particularly suitable as unsaturated sulfonic acids are compounds of the formula (II) 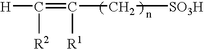
where n is 0 or 1 and R¹ and R², independently of one another, are H, C₁-C₄-alkyl, CN or COOH.

Examples of monomers of the formula (II) are vinylsulfonic acid and methallylsulfonic acid.

Aromatic unsaturated sulfonic acids, for example m-styrenesulfonic acid and/or p-styrenesulfonic acid, are furthermore suitable.

Examples of further suitable unsaturated sulfonic acids are arylamidopropanesulfonic acid 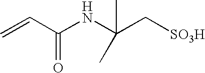
sulfoethyl methacrylate 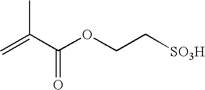
and
3-sulfopropyl acrylate 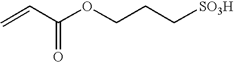
and 2-sulfopropyl acrylate.

Suitable unsaturated phosphonic acids are all compounds of the formula R—PO₃H, where R is a radical having at least one ethylenically unsaturated group.

Particularly suitable unsaturated phosphonic acids are compounds of the formula (III) 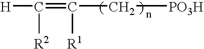
where n is 0 or 1 and R¹ and R², independently of one another, are H, C₁-C₄-alkyl, CN or COOH.

An example of a monomer of the formula (III) is vinylphosphonic acid.

Said monomers can be used individually or as a mixture.

The polymeric grafting base c) is selected from compounds of the formula

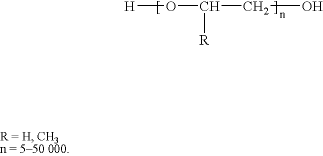

The molecular weight of the polyethers is from 200 to 2 500 000 (number average), preferably from 300 to 1 000 000, in particular from 400 to 100 000, particularly preferably from 500 to 50 000, very particularly preferably from 800 to 40 000.

Advantageously, homopolymers of ethylene oxide or copolymers containing from 40 to 99% by weight of ethylene oxide are used. For the ethylene oxide polymers preferably to be used, the amount of ethylene oxide incorporated in the form of polymerized units is therefore from 40 to 100 mol %. A suitable comonomer for these copolymers is propylene oxide. The ethylene oxide content of the copolymers is preferably from 40 to 99 mol % and the propylene oxide content from 1 to 60 mol %.

The grafting base c) is prepared by known processes, for example solution, precipitation, suspension or emulsion polymerization using compounds which form free radicals under the polymerization conditions. The polymerization temperatures are usually from 30 to 200° C., preferably from 40 to 110° C. Suitable initiators are, for example, azo and peroxy compounds and the conventional redox initiator systems, such as combinations of hydrogen peroxide and reducing compounds, for example sodium sulfite, sodium bisulfite, sodium formaldehyde sulfoxylate and hydrazine. These systems can, if appropriate, additionally contain small amounts of a heavy metal salt.

Chain transfer agents which may be used are the conventional compounds known to a person-skilled in the art, for example sulfur compounds (e.g. mercaptoethanol, 2-ethylhexyl thioglycolate, thioglycolic acid and dodecyl mercaptan), and tribromochloromethane or other compounds which have a regulating effect on the molecular weight of the polymers obtained.

If appropriate, thiol-containing silicone compounds may also be used.

Silicone-free chain transfer agents are preferably used.

In a preferred embodiment, the polymers are prepared in the presence of a crosslinking agent d).

Crosslinking monomers d) which may be used are compounds having at least two ethylenically unsaturated double bonds, for example esters of ethylenically unsaturated carboxylic acids, such as acrylic acid or methacrylic acid, and polyhydric alcohols, and ethers of at least dihydric alcohols, for example vinyl ether or allyl ether.

Examples of the parent alcohols are dihydric alcohols, such as 1,2-ethanediol, 1,2-propanediol, 1,3-propanediol, 1,2-butanediol, 1,3-butanediol, 2,3-butanediol, 1,4-butanediol, but-2-ene-1,4-diol, 1,2-pentanediol, 1,5-pentanediol, 1,2-hexanediol, 1,6-hexanediol, 1,10-decanediol, 1,2-dodecanediol, 1,12-dodecanediol, neopentylglycol, 3-methylpentane-1,5-diol, 2,5-dimethyl-1,3-hexanediol, 2,2,4-trimethyl-1,3-pentanediol, 1,2-cyclohexanediol, 1,4-cyclohexanediol, 1,4-bis(hydroxymethyl)cyclohexane, the monoester of neopentylglycol with hydroxypivalic acid, 2,2-bis(4-hydroxyphenyl)propane, 2,2-bis[4-(2-hydroxypropyl)phenyl]propane, diethylene glycol, triethylene glycol, tetraethylene glycol, dipropylene glycol, tripropylene glycol, tetrapropylene glycol, 3-thiopentane-1,5-diol, and polyethylene glycols, polypropylene glycols and polytetrahydrofurans having molecular weights of in each case from 200 to 10 000. In addition to the homopolymers of ethylene oxide or of propylene oxide, block copolymers of ethylene oxide or propylene oxide or copolymers which contain incorporated ethylene oxide and propylene oxide groups may also be used. Examples of parent alcohols having more than 2 OH groups are trimethylolpropane, glycerol, pentaerythritol, 1,2,5-pentanetriol, 1,2,6-hexanetriol, triethoxycyanuric acid, sorbitan, and sugars, such as sucrose, glucose and mannose. Of course, the polyhydric alcohols may also be used as a corresponding ethoxylate or propoxylate after reaction with ethylene oxide or propylene oxide. The polyhydric alcohols may also first be converted into the corresponding glycidyl ethers by reaction with epichlorohydrin.

Further suitable crosslinking agents d) are the vinyl esters or the esters of monohydric, unsaturated alcohols with ethylenically unsaturated C₃- to C₆-carboxylic acids, for example acrylic acid, methacrylic acid, itaconic acid, maleic acid or fumaric acid. Examples of such alcohols are allyl alcohol, 1-buten-3-ol, 5-hexen-1-ol, 1-octen-3-ol, 9-decen-1-ol, dicyclopentenyl alcohol, 10-undecen-1-ol, cinnamic alcohol, citronellol, crotyl alcohol or cis-9-octadecen-1-ol. However, the monohydric, unsaturated alcohols may also be esterified with monobasic carboxylic acids, for example malonic acid, tartaric acid, trimellitic acid, phthalic acid, terephthalic acid, citric acid or succinic acid.

Further suitable crosslinking agents d) are esters of unsaturated carboxylic acids with the polyhydric alcohols described above, for example of oleic acid, crotonic acid, cinnamic acid or 10-undecenoic acid.

In addition, straight-chain or branched, linear or cyclic aliphatic or aromatic hydrocarbons which have at least two double bonds which must not be conjugated in the case of the aliphatic hydrocarbons, e.g. divinylbenzene, divinyltoluene, 1,7-octadiene, 1,9-decadiene, 4-vinyl-1-cyclohexene, trivinylcyclohexane or polybutadienes having molecular weights of from 200 to 20 000, are also suitable.

Amides of unsaturated carboxylic acids, e.g. acrylic and methacrylic acid, itaconic acid and maleic acid, and N-allylamines of at least difunctional amines, such as 1,2-diaminomethane, 1,2-diaminoethane, 1,3-diaminopropane, 1,4-diaminobutane, 1,6-diaminohexane, 1,12-dodecanediamine, piperazine, diethylenetriamine or isophoronediamine, are furthermore suitable. Amides of allylamine and unsaturated carboxylic acids, such as acrylic acid, methacrylic acid, itaconic acid or maleic acid, or at least dihydric carboxylic acids, as described above, are furthermore suitable.

Triallylamine or corresponding ammonium salts, e.g. triallylmethylammonium chloride or methylsulfate, are furthermore suitable as crosslinking agents.

Furthermore, N-vinyl compounds of urea derivatives, at least difunctional amides, cyanurates or urethanes, for example of urea, ethyleneurea, propyleneurea or tartaramide, e.g. N,N′-divinylethyleneurea or N,N′-divinylpropyleneurea, may be used.

Further suitable crosslinking agents are divinyldioxane, tetraallylsilane or tetravinylsilane.

Particularly preferred crosslinking agents are, for example, methylenebisacrylamide, divinylbenzene, triallylamine and triallylammonium salts, divinylimidazole, N,N′-divinylethyleneurea, reaction products of polyhydric alcohols with acrylic acid or methacrylic acid, methacrylic esters and acrylic esters of polyalkylene oxides or polyhydric alcohols which have been reacted with ethylene oxide and/or propylene oxide and/or epichlorohydrin, and allyl or vinyl ethers of polyhydric alcohols, for example 1,2-ethanediol, 1,4-butanediol, diethylene glycol, trimethylolpropane, glycerol, pentaerythritol, sorbitan and sugars, such as sucrose, glucose or mannose.

Very particularly preferred crosslinking agents are pentaerythrityl triallyl ether, allyl ethers of sugars, such as sucrose, glucose or mannose, divinylbenzene, methylenebisacrylamide, N,N′-divinylethyleneurea and (meth)acrylic esters of glycol, butanediol, trimethylolpropane or glycerol or (meth)acrylic esters of glycol, butanediol, trimethylolpropane or glycerol which have been reacted with ethylene oxide and/or epichlorohydrin.

The amount of crosslinking monomers is from 0 to 10, preferably from 0 to 5, very particularly preferably from 0 to 2, %, by weight.

In the polymerization for the preparation of the novel polymers, other polymers, for example polyamides, polyurethanes, polyesters, homo- and copolymers of ethylenically unsaturated monomers, may, if appropriate, also be present. Examples of such polymers, some of which are also used in cosmetics, are the polymers known under the trade names Amerhold™, Ultrahold™, Ultrahold Strong™, Luviflex™ VBM, Luvimer™, Acronal™, Acudyne™, Stepanhold™, Lovocryl™, Versatyl™, Amphomer™ or Eastma AQ™.

For example, mineral bases, such as sodium carbonate, alkali metal hydroxides and ammonia, organic bases, such as amino alcohols, especially 2-amino-2-methyl-1-propanol, monoethanolamine, diethanolamine, triethanolamine, triisopropanolamine, tri[(2-hydroxy)-1-propyl]amine, 2-amino-2-methyl-1,3-propanediol, 2-amino-2-hydroxymethyl-1,3-propanediol and diamines, such as lysine, may be used as neutralizing agents for monomers carrying acid groups.

For the preparation of the polymers, the monomers of component a) and of component b) can be polymerized in the presence of the grafting base c), both with the aid of free radical initiators and by the action of high-energy radiation, which is also to be understood as meaning the action of high-energy electrons.

The peroxo and/or azo compounds customary for this purpose may be used as initiators for the free radical polymerization, for example alkali metal or ammonium peroxodisulfates, diacetyl peroxide, dibenzoyl peroxide, succinyl peroxide, di-tert-butyl peroxide, tert-butyl perbenzoate, tert-butyl perpivalate, tert-butyl peroxy-2-ethylhexanoate, tert-butyl permaleate, cumyl hydroperoxide, diisopropyl peroxodicarbamate, bis(o-toluoyl) peroxide, didecanoyl peroxide, dioctanoyl peroxide, dilauroyl peroxide, tert-butyl perisobutyrate, tert-butyl peracetate, di-tert-amyl peroxide, tert-butyl hydroperoxide, azobisisobutyronitrile, azobis(2-amidinopropane) dihydrochloride or 2,2′-azobis(2-methylbutyronitrile). Initiator mixtures or redox initiator systems, such as ascorbic acid/iron(II) sulfate/sodium peroxodisulfate, tert-butyl hydroperoxide/sodium disulfite, and tert-butyl hydroperoxide/sodium hydroxymethanesulfonate, are also suitable.

Organic peroxides are preferably used.

The polymerization can also be carried out by the action of ultraviolet radiation, if appropriate in the presence of UV initiators. For the polymerization under the action of UV radiation, the suitable photoinitiators or sensitizers customary for this purpose are used. These are, for example, compounds such as benzoin and benzoin ethers, α-methylbenzoin or α-phenylbenzoin. Triplet sensitizers, such as benzyl diketals, can also be used. UV radiation sources used, in addition to high-energy UV lamps, such as carbon arc lamps, mercury vapor lamps or xenon lamps, are also, for example, low-UV light sources, such as fluorescent tubes having a high blue content.

The amounts of initiator or initiator mixtures used are from 0.01 to 10, preferably from 0.1 to 5, % by weight, based on monomer used.

The polymerization is effected at from 40 to 200° C., preferably from 50 to 140° C., particularly preferably from 60 to 110° C. It is usually carried out under atmospheric pressure but may also take place under reduced or superatmospheric pressure, preferably from 1 to 5 bar.

The polymerization can be carried out, for example, as solution polymerization, mass polymerization, emulsion polymerization, inverse emulsion polymerization, suspension polymerization, inverse suspension polymerization or precipitation polymerization, without the methods which can be used being restricted thereto. The polymerization is particularly preferably carried out as precipitation polymerization.

In the mass polymerization, it is possible to adopt a procedure in which the grafting base c) is dissolved in the monomer of group a) (N-vinylformamide) and further comonomers of group b) and, after addition of a polymerization initiator, the mixture is polymerized. The polymerization can also be carried out semicontinuously by first initially taking a part, for example 10%, of the mixture to be polymerized and comprising the grafting base c), the monomer a), further comonomers of group b) and initiator, heating the mixture to the polymerization temperature and, after initiation of the polymerization, adding the remainder of the mixture to be polymerized according to the progress of the polymerization. The polymers can also be obtained by initially taking the grafting base c) in a reactor and heating it to the polymerization temperature and adding the monomer of group a), further comonomers of group b) and polymerization initiator either all at once, batchwise or, preferably, continuously and effecting polymerization.

If desired, the polymerization described above can also be carried out in a solvent. Suitable solvents are, for example, alcohols, such as methanol, ethanol, n-propanol, isopropanol, n-butanol, sec-butanol, tert-butanol, n-hexanol and cyclohexanol, and glycols, such as ethylene glycol, propylene glycol and butylene glycol, and the methyl or ethyl ethers of dihydric alcohols, diethylene glycol, triethylene glycol, glycerol and dioxane. The polymerization can also be carried out in water as a solvent. In this case, a solution is initially present, which is more or less soluble in water depending on the amount of added monomers of component a). In order to dissolve water-insoluble products which may form during polymerization, for example organic solvents, such as monohydric alcohols of 1 to 3 carbon atoms, acetone or dimethylformamide, may be added. However, in the polymerization in water, it is also possible to adopt a procedure in which the water-insoluble polymers are converted into finely divided dispersions by adding conventional emulsifiers or protective colloids, e.g. polyvinyl alcohol.

Emulsifiers used are, for example, ionic or nonionic surfactants whose HLB value is from 3 to 13. For the definition of the HLB value, reference is made to the publication by W. C. Griffin, J. Soc. Cosmetic Chem. 5 (1954), 249.

The amount of surfactants is from 0.1 to 10% by weight, based on the polymer. When water is used as a solvent, solutions or dispersions of the polymers are obtained. If solutions of the polymer in an organic solvent or in mixtures of an organic solvent and water are prepared, from 5 to 2000, preferably from 10 to 500, parts by weight of the organic solvent or of the solvent mixture are used per 100 parts by weight of-the polymer.

Polymers which are obtainable by free radical graft copolymerization of
10-90% by weight of N-vinylformamide
1-60% by weight of one or more copolymerizable monomers b), in particular of the monomers of the formula (I)
10-80% by weight of polymeric grafting base c), in particular having a molecular weight of from 500 to 50 000
0-10% by weight of one or more crosslinking monomers d),
with the proviso that the amounts sum to 100%, are preferred.

Polymers which are obtainable by free radical graft copolymerization of
20-80% by weight of N-vinylformamide
10-60% by weight of one or more copolymerizable monomers b), in particular of the monomers of the formula (I)
20-70% by weight of polymeric grafting base c), in particular having a molecular weight of from 500 to 50 000
0-5% by weight of one or more crosslinking monomers d),
with the proviso that the amounts sum to 100%, are preferred.

Polymers which are obtainable by free radical graft copolymerization of
40-80% by weight of N-vinylformamide
10-40% by weight of one or more copolymerizable monomers b), in particular of the monomers of the formula (I)
20-80% by weight of polymeric grafting base c), in particular having a molecular weight of from 500 to 50 000
0-2% by weight of one or more crosslinking monomers d),
with the proviso that the amounts sum to 100%, are very particularly preferred.

The preferred method for the preparation of the novel polymer is precipitation polymerization. Solvents in which the starting materials for the polymerization are soluble and the polymer formed is insoluble are used for this polymerization. Suitable solvents are, for example, aromatic hydrocarbons, such as toluene, xylenes, benzene or aliphatic hydrocarbons, such as n-alkanes, cyclohexane, esters of acetic acid, such as ethyl acetate or butyl acetate, ethers, such as diethyl ether, dipropyl ether, dibutyl ether, methyl tert-butyl ether or diethylene glycol dimethyl ether, ketones, such as acetone or methyl ethyl ketone, and-mixtures of these solvents. Mixtures of, for example, ethyl acetate and butyl acetate are particularly suitable since, in this solvent mixture, the polymers are obtained in a form which can be easily isolated (settling is accelerated) and moreover the reaction temperature in the mixtures of butyl acetate and ethyl acetate can be chosen to be above the boiling point of ethyl acetate with simultaneous evaporative cooling by boiling ethyl acetate.

Initiators which may be used are all initiators as also used in solution polymerization. Preferably from 0.01 to 1.5% by weight, based on the monomers used, of initiator are used.

The precipitation polymerization is usually carried out at from 20 to 150° C., preferably from 40 to 120° C., in particular from 60 to 100° C.

The precipitation polymerization is usually carried out at from 1 to 15, in particular from 1 to 6, bar.

Through the corresponding boiling points, the solvent or solvent mixture determines the maximum reaction temperature if polymerization is effected at atmospheric pressure. However, polymerization under pressure is also feasible.

In general, the precipitation polymerization can be carried out at solids contents up to about 40%. A range of from 25 to 40% is preferred. Particularly at high solids contents, it is advisable to carry out the polymerization in the presence of a protective colloid polymer. Suitable protective colloid polymers are those which are readily soluble in the solvents used and do not react with the monomers. Suitable polymers are, for example, copolymers of maleic acid with vinyl alkyl ethers and/or olefins of 8 to 20 carbon atoms or corresponding copolymers of maleic monoesters with C10-C20 alcohols or mono- and diamides of maleic acid with C10-C20 alkylamines and polyvinyl alcohol ethers having alkyl groups which carry 1 to 20 carbon atoms, or polyvinyl methyl, ethyl, isobutyl or octadecyl ether. The amount of protective colloid polymer used is usually from 0.05 to 4, preferably from 0.1 to 2, % by weight (based on monomers). It is often advantageous to use mixtures of a plurality of protective colloid polymers.

The polymerization is carried out by initially taking solvent, grafting base, protective colloid polymer and possibly crosslinking agent, heating them, and carrying out the polymerization by adding initiator and monomers (possibly dissolved in the same solvent or solvent mixture). However, portions of the monomers and of the initiator may also be initially taken (e.g. 10%), this mixture heated to the polymerization temperature and, after initiation of the reaction, the remainder of the mixture to be polymerized added according to the progress of the polymerization. It is also possible for the crosslinking agent used to be initially taken in portions or not to be initially taken at all and the remainder to be added together with the remaining components. At relatively low solids contents, it is also conceivable initially to take all starting materials in one batch reaction.

Monomer and initiator are generally metered in in a time of from 1 to 10, preferably from 2 to 5, hours.

The precipitated polymer is then isolated from the reaction mixture, for which purpose any general method for isolating the polymers in the conventional precipitation polymerization can be used. Such methods are filtration, centrifuging, evaporation of the solvent or combinations of these methods. For further purification of the polymer to eliminate unpolymerized components, the polymer is washed. For this purpose, it is possible in principle to use the same solvents as are suitable for the polymerization. For easier drying of the polymers, however, it is advisable to use low-boiling solvents, such as acetone.

The novel graft copolymers can be hydrolyzed after the polymerization. By means of the hydrolysis, a cationic group is produced in the polymer. This may lead to increased water solubility and improved conditioning properties in cosmetic applications.

From the graft copolymers described above, units of the formula (V) 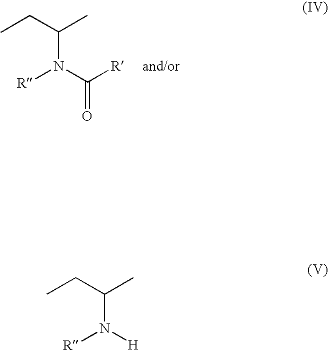
are obtained by partial or complete elimination of the formyl groups from the N-vinylformamide incorporated into the polymer, with formation of amino or ammonium groups.

In the formulae (IV) and (V), the substituents R′ and R″ each have the abovementioned meanings. Depending on the reaction conditions chosen in the hydrolysis, either partial or complete hydrolysis of the units (IV) is obtained.

If, in addition to the vinylpyrrolidone units insensitive to hydrolysis, the grafting base also contains comonomers which are sensitive to hydrolysis, e.g. vinyl acetate or acrylamide, hydrolysis also takes place in the grafting base. Thus, vinyl acetate reacts to give vinyl alcohol groups and acrylamide to give acrylic acid groups.

Suitable hydrolyzing agents are mineral acids, such as hydrogen halides, which can be used in gaseous form or in aqueous solution. Hydrochloric acid, sulfuric acid, nitric acid and phosphoric acid and organic acids, such as C₁- to C₅-carboxylic acids, and aliphatic or aromatic sulfonic acids are preferably used. From 0.05 to 2, preferably from 1 to 1.5, mole equivalents of an acid are required per formyl group equivalent which is to be eliminated from the polymerized units (IV).

The hydrolysis of the polymerized units of the structure (IV) can also be carried out with the aid of bases, for example of metal hydroxides, in particular of alkali metal and alkaline earth metal hydroxides. Sodium hydroxide or potassium hydroxide is preferably used. The hydrolysis can, if appropriate, also be carried out in the presence of ammonia or amines.

The hydrolysis in the acidic or in the alkaline pH range is effected, for example, at from 30 to 170° C., preferably from 50 to 120° C. It is complete after from about 2 to 8, preferably from 3 to 5, hours. After these reaction times, degrees of hydrolysis of the units of the polymerized monomers of the formula (I) of from 1 to 100% are achieved. A procedure in which the bases or acids are added in aqueous solution for the hydrolysis has proven particularly useful. After the hydrolysis, a neutralization is generally carried out so that the pH of the hydrolyzed polymer solution is from 2 to 8, preferably from 3 to 7. The neutralization is required when progress of the hydrolysis of partly hydrolyzed polymers is to be avoided or retarded. The hydrolysis can also be carried out with the aid of enzymes.

The polymers thus prepared can subsequently be cationized by reaction of hydroxyl and/or amino functions present in the polymer with epoxides of the formula X (R³¹═C₁- to C₄₀-alkyl).

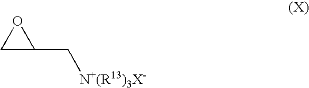

The hydroxyl groups of the polyvinyl alcohol units and vinylamine units, formed by hydrolysis of vinylformamide, can preferably be reacted with the epoxides.

The epoxides of the formula x may also be produced in situ by reacting the corresponding chlorohydrins with bases, for example sodium hydroxide. 2,3-Epoxypropyltrimethylammonium chloride or 3-chloro-2-hydroxypropyltrimethylammonium chloride is preferably used.

The K values of the polymers should be from 10 to 300, preferably from 25 to 250, particularly preferably from 25 to 200, very particularly preferably from 30 to 150. The K value desired in each case can be established in a manner known per se by the composition of the starting materials. The K values are determined according to Fikentscher, Cellulosechemie 13 (1932), 58 to 64 and 71 to 74, in N-methylpyrrolidone at 25° C. and polymer concentrations which, depending on the K value range, are from 0.1 to 5% by weight.

For removing solvents, the polymer solutions can be subjected to steam distillation. After the steam distillation, aqueous solutions or dispersions are obtained, depending on the choice of components.

The polymers obtained can also be postcrosslinked by reacting the hydroxyl groups or amino groups in the polymer with at least bifunctional reagents. Water-soluble products are obtained in the case of low degrees of crosslinking, and water-swellable or insoluble products in the case of high degrees of crosslinking. For example, the novel polymers can be reacted with dialdehydes and diketones, e.g. glyoxal, glutaraldehyde, succinaldehyde or terephthalaldehyde. Furthermore, aliphatic or aromatic carboxylic acids, for example maleic acid, oxalic acid, malonic acid, succinic acid or citric acid, or carboxylic acid derivatives, such as carboxylic esters, anhydrides or halides, are suitable. Polyfunctional epoxides, e.g. epichlorohydrin, glycidyl methacrylate, ethylene glycol diglycidyl ether, 1,4-butanediol diglycidyl ether or 1,4-bis(glycidyloxy)benzene, are furthermore suitable. Diisocyanates, for example hexamethylene diisocyanate, isophorone diisocyanate, methylenediphenyl diisocyanate or toluene diisocyanate, or divinyl sulfone are furthermore suitable.

Inorganic compounds, such as boric acid or boric acid salts, for example sodium metaborate, borax (disodium tetraborate), and salts of polyvalent cations, e.g. copper(II) salts, such as copper(II) acetate, or zinc, aluminum or titanium salts, are furthermore suitable.

Boric acid or boric acid salts, such as sodium metaborate or disodium tetraborate, are preferably suitable for postcrosslinking. The boric acid or boric acid salts, preferably as salt solutions, can be added to the solutions of the novel polymers. The boric acid or boric acid salts are preferably added to the aqueous polymer solutions.

The boric acid or boric acid salts can be added to the polymer solutions directly after the preparation. However, it is also possible to add the boric acid or boric acid salts subsequently to the cosmetic formulations with the novel polymers or during the preparation process of the cosmetic formulations.

The amount of boric acid or boric acid salts is from 0 to 15, preferably from 0 to 10, particularly preferably from 0 to 5, % by weight, based on the novel polymers.

The polymer solutions and dispersions can be converted into powder form by various drying methods, e.g. spray drying, fluidized spray drying, drum drying or freeze drying. A preferably used drying method is spray drying. An aqueous solution or dispersion can be prepared again from the resulting dry polymer powder by dissolving or redispersing in water. The conversion into powder form has the advantage of better storability, possibility of easier transport and less tendency to microorganism attack.

The novel water-soluble or water-dispersible graft copolymers are very suitable for use in cosmetic formulations, in particular as thickeners.

It has been found that the novel polymers have good hair setting properties and at the same time are good gel formers. They are therefore particularly suitable in thickened cosmetic formulations (e.g. gels) and can be used in them also without the addition of conventional thickeners.

The novel polymers are suitable as styling compositions and/or conditioning compositions in cosmetic hair formulations, such as hair repair treatments, hair lotions, hair rinses, hair emulsions, damaged end fluids, neutralizing compositions for permanent waves, hot oil treatment preparations, conditioners, setting lotions or hair sprays. Depending on the field of use, the cosmetic hair formulations can be applied as spray, foam, gel, gel spray or mousse.

Use in gel or gel spray is preferred.

In a preferred embodiment, the novel cosmetic hair formulations contain
a) 0.05-20% by weight of the novel polymer
b) 20-99.95% by weightof water and/or alcohol
c) 0-79.5% by weight of further components.

Alcohol is to be understood as meaning all alcohols customary in cosmetics, e.g. ethanol, isopropanol and n-propanol.

Further components are to be understood as meaning the additives customary in cosmetics, for example propellants, antifoams, surface-active compounds, i.e. surfactants, emulsifiers, foam formers and solubilizers. The surface-active compounds used may be anionic, cationic, amphoteric or neutral. Further conventional components may moreover be, for example, preservatives, perfume oils, opacifiers, active ingredients, UV filters, care substances, such as panthenol, collagen, vitamins, protein hydrolysis products, alpha- and beta-hydroxycarboxylic acids, stabilizers, pH regulators, dyes, viscosity regulators, gel formers, salts, moisturizers, refatting agents and further conventional additives.

These also include all styling and conditioner polymers which are known in cosmetics and may be used in combination with the novel polymers if very special properties are to be established.

Suitable conventional polymers for hair cosmetics are, for example, anionic polymers. Such anionic polymers are homo- and copolymers of acrylic acid and methacrylic acid or the salts thereof, copolymers of acrylic acid and acrylamide and the salts thereof; sodium salts of polyhydroxycarboxylic acids, water-soluble or water-dispersible polyesters, polyurethanes (Luviset® P.U.R.) and polyureas. Particularly suitable polymers are copolymers of tert-butyl acrylate, ethyl acrylate, methacrylic acid (e.g. Luvimer® 100P), copolymers of N-tert-butylacrylamide, ethyl acrylate, acrylic acid (Ultrahold® 8, strong), copolymers of vinyl acetate, crotonic acid and, if appropriate, further vinyl esters (e.g. Luviset® grades), maleic anhydride copolymers, if appropriate reacted with alcohols, anionic polysiloxanes, e.g. carboxyfunctional copolymers of vinylpyrrolidone, tert-butyl acrylate or methacrylic acid (e.g. Luviskol® VBM).

Acrylates having an acid number greater than or equal to 120 and copolymers of tert-butyl acrylate, ethyl acrylate and methacrylic acid are very particularly preferred as anionic polymers.

Further suitable polymers for hair cosmetics are cationic polymers having the designation polyquaternium according to INCI, e.g. copolymers of vinylpyrrolidone/N-vinylimidazolium salts (Luviquat® FC, Luviquat® HM, Luviquat® MS, Luviquat® Care), copolymers of N-vinylpyrrolidone/dimethylaminoethyl methacrylate, quaternized with diethyl sulfate (Luviquat® PQ 11), copolymers of N-vinylcaprolactam, N-vinylpyrrolidone/N-vinylimidazolium salts (Luviquat® Hold); cationic cellulose derivatives (polyquaternium-4 and -10), acrylamide copolymers (polyquaternium-7).

Neutral polymers, such as polyvinylpyrrolidones, copolymers of N-vinylpyrrolidone and vinyl acetate and/or vinyl propionate, polysiloxanes, polyvinylcaprolactam and copolymers with N-vinylpyrrolidone, polyethylenimines and salts thereof, polyvinylamines and salts thereof, cellulose derivatives, polyaspartic acid salts and derivatives, are also suitable as further polymers for hair cosmetics.

For establishing certain properties, the formulations may additionally contain conditioning substances based on silicone compounds. Suitable silicone compounds are, for example, polyalkylsiloxanes, polyarylsiloxanes, polyarylalkylsiloxanes, polyethersiloxanes, silicone resins or dimethicone copolyols (CTFA) and amino functional silicone compounds, such as amodimethicone (CTFA).

The novel polymers are particularly suitable as setting compositions in hairstyling formulations, in particular hair sprays (aerosol sprays and pump sprays without propellant) and hair foams (aerosol foams and pump foams without propellant), in particular in gels and gel sprays.

In a preferred embodiment, these formulations contain
a) 0.1-10% by weight of the novel polymer
b) 20-99.9% by weight of water and/or alcohol
c) 0-70% by weight of a propellant
d) 0-20% by weight of further components.

Propellants are the propellants usually used for hair sprays or aerosol foams. Mixtures of propane/butane, pentane, dimethyl ether, 1,1-difluoroethane (HFC-152 a), carbon dioxide, nitrogen or compressed air are suitable.

A formulation preferred according to the invention for aerosol hair foams contains
a) 0.1-10% by weight of the novel polymer
b) 55-94.8% by weight of water and/or alcohol
c) 5-20% by weight of a propellant
d) 0.1-5% by weight of an emulsifier
e) 0-10% by weight of further components.

Emulsifiers which may be used are all emulsifiers usually used in hair foams. Suitable emulsifiers may be nonionic, cationic or anionic.

Examples of nonionic emulsifiers (INCI nomenclature) are laureths, e.g. laureth-4; ceteths, e.g. cetheth-1, polyethylene glycol cetyl ether; ceteareths, e.g. cetheareth-25, polyglycol fatty acid glycerides, hydroxylated lecithin, lactyl esters of fatty acids, alkylpolyglycosides.

Examples of cationic emulsifiers are cetyldimethyl-2-hydroxyethylammonium dihydrogen phosphate, cetyltrimonium chloride, cetyltrimonium bromide, cocotrimonium methylsulfate, quaternium-1 bis x (INCI).

Anionic emulsifiers may be selected, for example, from the group consisting of the alkylsulfates, alkyl ether sulfates, alkanesulfonates, alkylarylsulfonates, alkylsuccinates, alkylsulfosuccinates, N-alkoylsarcosinates, acyltaurates, acylisethionates, alkylphosphates, alkyl ether phosphates, alkyl ether carboxylates, alpha-olefinsulfonates, in particular the alkali metal and alkaline earth metal salts, e.g. sodium, potassium, magnesium, calcium and ammonium and triethanolamine salts. The alkyl ether sulfates, alkyl ether phosphates and alkyl ether carboxylates may have from 1 to 10 ethylene oxide or propylene oxide units, preferably from 1 to 3 ethylene oxide units, in the molecule.

Owing to their thickening effect, the novel polymers can be used as the sole gel former in cosmetic formulations. Moreover, they are also suitable for use in combination with conventional gel formers.

Surprisingly, it is possible to obtain clear gels.

A formulation suitable according to the invention for styling gels may have, for example, the following composition:
a) 0.1-10% by weight of the novel polymer
b) 60-99.85% by weight of water and/or alcohol
c) 0.05-10% by weight of a gel former
d) 0-20% by weight of further components.

Gel formers which may be used are all gel formers customary in cosmetics. These include readily crosslinked polyacrylic acid, for example carbomer (INCI), cellulose derivatives, e.g. hydroxypropylcellulose, hydroxyethylcellulose, cationically modified celluloses, polysaccharides, e.g. xanthum gum, caprylic/capric triglycerides, sodium acrylate copolymer, polyquaternium-32 (and) liquid paraffin (INCI), sodium acrylate copolymer (and) liquid paraffin (and) PPG-1 trideceth-6, acrylamidopropyl trimonium chloride/acrylamide copolymer, steareth-10 allyl ether acrylate copolymer, polyquaternium-37 (and) liquid paraffin (and) PPG-1 trideceth-6, polyquaternium-37 (and) propylene glycol dicaprate dicaprylate (and) PPG-1 trideceth-6, polyquaternium-7, polyquaternium-44.

The novel polymers can also be used in shampoo formulations as setting and/or conditioning compositions.

Preferred shampoo formulations contain
a) 0.05-10% by weight of the novel polymer
b) 25-94.95% by weightof water
c) 5-50% by weight of surfactants
d) 0-5% by weight of a further conditioning composition
e) 0-10% by weight of further cosmetic components.

All anionic, neutral, amphoteric or cationic surfactants usually used in shampoos can be used in the shampoo formulations.

Suitable anionic surfactants are, for example, alkylsulfates, alkyl ether sulfates, alkanesulfonates, alkylarylsulfonates, alkylsuccinates, alkylsulfosuccinates, N-alkoylsarcosinates, acyltaurates, acylisethionates, alkylphosphates, alkyl ether phosphates, alkyl ether carboxylates, alpha-olefinsulfonates, in particular the alkali metal and alkaline earth metal salts, e.g. sodium, potassium, magnesium, calcium and ammonium and triethanolamine salts. The alkyl ether sulfates, alkyl ether phosphates and alkyl ether carboxylates may have from 1 to 10 ethylene oxide or propylene oxide units, preferably from 1 to 3 ethylene oxide units, in the molecule.

For example, sodium laurylsulfate, ammonium laurylsulfate, sodium lauryl ether sulfate, ammonium lauryl ether sulfate, sodium lauroylsarcosinate, sodium oleylsuccinate, ammonium laurylsulfosuccinate, sodium dodecylbenzenesulfonate and triethanolaminedodecylbenzenesulfonate are suitable.

Suitable amphoteric surfactants are, for example, alkylbetaines, alkylamidopropylbetaines, alkylsulfobetaines, alkylglycinates, alkylcarboxyglycinates, alkylamphoacetates or -propionates, alkylamphodiacetates or -dipropionates.

For example, cocodimethylsulfopropylbetaine, laurylbetaine, cocamidopropylbetaine or sodium cocamphopropionate may be used.

Suitable nonionic surfactants are, for example, the reaction products of aliphatic alcohols or alkylphenols having 6 to 20 carbon atoms in the alkyl chain, which may be linear or branched, with ethylene oxide and/or propylene oxide. The amount of alkylene oxide is from-about 6 to 60 moles per mole of alcohol. Furthermore, alkylamine oxides, mono- or dialkylalkanolamides, fatty esters of polyethylene glycols, alkylpolyglycosides or sorbitan ether esters are suitable.

The shampoo formulations may also contain conventional cationic surfactants, e.g. quaternary ammonium compounds, for example cetyltrimethylammonium chloride.

In the shampoo formulations, conventional conditioning compositions can be used in combination with the novel polymers for achieving certain effects. These include, for example, cationic polymers having the designation polyquaternium according to INCI, in particular copolymers of vinylpyrrolidone/N-vinylimidazolium salts (Luviquat® FC, Luviquat® HM, Luviquat® MS, Luviquat® Care), copolymers of N-vinylpyrrolidone/dimethylaminoethyl methacrylate, quaternized with diethyl sulfate (Luviquat® PQ 11), copolymers of N-vinylcaprolactam/N-vinylpyrrolidone/N-vinylimidazolium salts (Luviquat® Hold); cationic cellulose derivatives (polyquaternium-4 and -10), acrylamide copolymers (polyquaternium-7). It is furthermore possible to use protein hydrolysis products, and conditioning substances based on silicone compounds, for example polyalkylsiloxanes, polyarylsiloxanes, polyarylalkylsiloxanes, polyethersiloxanes or silicone resins. Further suitable silicone compounds are dimethicone copolyols (CTFA) and amino functional silicone compounds, such as amodimethicone (CTFA).

### EXAMPLES

1. General Preparation Method for Precipitation Polymers Examples 1 to 7

In a stirred reactor having a nitrogen feed, reflux condenser and metering apparatus, the initially taken mixture is heated to 87.5° C. under nitrogen. Feeds 1 and 2 are then metered in in 4 hours, and feed 3 in 5 hours (⅓ in 3 hours and ⅔ in 2 hours). Heating to 95° C. is then effected and polymerization is carried out for 2 hours at this temperature. After the end of the reaction, cooling to room temperature is effected, dilution is effected with feed 4 and the precipitated polymer is filtered off, washed with acetone, sucked dry and dried in a vacuum drying oven (reduced pressure from a water jet pump) at 75° C.

All polymers are isolated in yields of >95%.

| | Example | | | | | | |
|---|---|---|---|---|---|---|---|
| | 1 | 2 | 3 | 4 | 5 | 6 | 7 |
| Initially taken mixture | | | | | | | |
| n-Butyl acetate/Ethyl | 600 | 540 | 540 | 540 | 540 | 540 | 540 |
| acetate | | | | | | | |
| (weight ratio 2:1) | | | | | | | |
| Polyethylene glycol | 105.6 | 108 | 108 | 108 | 108 | 108 | 108 |
| (M = 35 000) | | | | | | | |
| Polyvinylethyl ether | 13.2 | 13.5 | 13.5 | 13.5 | 13.5 | 13.5 | 13.5 |
| K value (5% in THF acc. | | | | | | | |
| to ISO1628-1): 10-17 | | | | | | | |
| (40% strength solution in | | | | | | | |
| butyl acetate) | | | | | | | |
| Pentaerythrityl triallyl | 1.48 | 2.16 | | 2.16 | | 2.16 | 2.16 |
| ether | | | | | | | |
| (technical-grade, Aldrich) | | | | | | | |
| Feed 3 | 8 | 7 | 7 | 7 | 7 | 7 | 7 |
| Feed 1 | | | | | | | |
| n-Butyl acetate/Ethyl | 197.12 | 99 | 99 | 99 | 99 | 76.5 | 96 |
| acetate | | | | | | | |
| N-vinylformamide | 253.44 | 270 | 270 | 270 | 270 | 202.5 | 288 |
| Stearyl methacrylate | 42.24 | 27 | 27 | 27 | 27 | 27 | |
| Feed 2 | | | | | | | |
| n-Butyl acetate/Ethyl | 84.48 | 135 | 135 | 135 | 135 | 202.5 | 144 |
| acetate | | | | | | | |
| (weight ratio 2:1) | | | | | | | |
| Acrylic acid (AS) | 126.72 | 135 | 135 | 135 | 135 | 202.5 | 144 |
| Feed 3 | | | | | | | |
| n-Butyl acetate/Ethyl | 156.83 | 156.76 | 156.76 | 158.38 | 158.38 | 156.76 | 156.76 |
| acetate | | | | | | | |
| tert-Butyl peroctanoate | 3.17 | 3.24 | 3.24 | 1.62 | 1.62 | 3.24 | 3.24 |
| Feed 4 | | | | | | | |
| Acetone | 500 | 500 | 500 | 500 | 500 | 500 | 500 |

2. Preparation Method for the Aqueous Solutions Neutralized with AMP

8 g of polymer (in each case from examples 1 to 7) are initially taken in a reaction flask having a plane-ground joint and having an anchor stirrer, internal thermometer, condenser and oil bath, 0.1%, based on the total batch, of the preservative Euxyl® K 100 (solution of isothiazolinone derivatives and benzyl alcohol, from Schülke & Mayr) and 400 g of water are added and heating to 80° C. is effected with stirring. The calculated amount of base 2-amino-2-methylpropan-1-ol (AMP) is mixed with 200 g of water and added to the polymer suspension and stirring is continued at. 80° C. until the polymer has dissolved. Thereafter, the remaining amount of water for a total batch of 800 g is stirred in and the warm solution is filled.

3. Viscosity Determination

The viscosity was determined according to Brookfield at 25° C. (Brookfield viscometer LVDV-II+).

| | AMP | | | |
|---|---|---|---|---|
| | (10% strength | | Viscosity | |
| Example No. | in water) in g | Spindle | Speed | CP |
| 1 | | 3 | 6 | 12897 |
| 2 | 24.4 | 4 | 30 | 12257 |
| 3 | 24.5 | 1 | 30 | 44.4 |
| 4 | 24.4 | 4 | 6 | 68278 |
| 5 | 24.5 | 1 | 30 | 23.4 |
| 6 | 36.6 | 4 | 30 | 7138 |
| 7 | 26.1 | 4 | 30 | 6259 |

4. Carbopol Compatibility:

Starting materials: AMP-neutralized solution of the polymers according to the above method, 1% strength carbopol solution (Carbopol 940), triethanolamine.

Procedure:

100 g of the polymer solution are weighed into a 250 ml wide-necked glass bottle, and 98.68 g of 1% strength aqueous solution of Carbopol 940 are added and thorough mixing is carried out. Thereafter, 1.32 g of triethanolamine are added dropwise, thorough mixing is carried out once again and the gel obtained or the solution is assessed.

| | | Gel: neutralized |
|---|---|---|
| | AMP-neutralized | polymer solution |
| | polymer solution | with carbopol |
| Example No. | (see under 2) | solution (see under 4) |
| 1 | − | ++ |
| 2 | ++ | ++ |
| 4 | ++ | ++ |
| 6 | − | ++ |
| 7 | − | ++ |

Rating:
virtually clear solution (+++)>slightly turbid solution (++)>fairly turbid solution (+)>turbid solution (−)
   5. Setting Effect

Batch.: 50 g of finished gel (neutralized with 1% strength AMP) 170 g of distilled water

Solids content of about 0.23%.

Bending Test

The dry, weighed hair strands are drawn 3 times through the dilute gel. After each passage, the excess is scraped off with the fingers. After the last wetting, the hair strand is pressed off between filter papers. The moist hair strand is weighed and then shaped in such a way that it gives a round section. The amount applied is from 1.00 to 1.30 g (depending on viscosity). After drying overnight in a conditioned chamber (20° C., 65% relative humidity), the bending test value is measured using a tensile tester (from Franck).

Curl Retention:

The dry hair strand is drawn 3 times through the dilute gel. After each passage, the excess is scraped off with the fingers. After the last wetting, the uniformly moistened hair strand is combed and is wound on Teflon rods (Ø 12 mm). From time to time, the hair strand is combed again. The hair strand is set by means of a filter paper strip and rubber rings. The curls are dried overnight at 70° C. After cooling to room temperature, the paper and the rubber rings are removed and the hair strand is carefully skimmed off the Teflon rod.

The initial length and the length after treatment in a conditioning chamber for 5 hours are determined and the curl retention is calculated.

| Example No. | Bending test (cN) | Curl retention (%) |
|---|---|---|
| 2 | 88 | 91 |
| 4 | 107 | 85 |
| 7 | 120 | 90 |

| | Bending | Curl |
|---|---|---|
| Polymers | test (cN) | retention (%) |
| Vinylpyrrolidone/Vinyl | 59 | 31 |
| acetate copolymer available | | |
| under the trade name | | |
| Luviskol ® VA64 P | | |
| Polyvinylpyrrolidones | 74 | 27 |
| available under the trade | | |
| name Luviskol ® K30 | | |

### Use Examples

The following formulations were each prepared using the 7 novel polymers.

### Examples 1 to 7

### Formulation for Aerosol Hair Foam

2.00% of copolymer from examples 1 to 7
2.00% of Luviquat ® Mono LS (coco trimonium methyl sulfate)
67.7% of water
10.0% of propane/butane 3.5 bar (20° C.)
q.s. perfume oil

### Comparative Example

2.00% polymer content of Luviquat Hold (polyquaternium-46)

2.00% of Luviquat® Mono LS (coco trimonium;methyl sulfate)

67.7% of water

10.0% of propane/butane 3.5 bar (20° C.)

q.s. perfume oil

### Examples 8 to 14

### Aerosol Hair Foam

INCI

| | |
|---|---|
| 4.00% of copolymer from examples 1 to 7 | |
| 0.20% of Cremophor ® A 25 | ceteareth-25 |
| 1.00% of Luviquat ® Mono CP | hydroxyethyl cetyldi- |
| | monium phosphate |
| 5.00% of ethanol | |
| 1.00% of panthenol | |
| 10.0% of propane/butane 3.5 bar (20° C.) | |
| q.s. perfume oil | |
| to 100% water | |

### Examples 15 to 21

### Pump Foams

| | |
|---|---|
| 2.00% of copolymer from examples 1 to 7 | |
| 2.00% of Luviflex ® Soft (polymer content) | |
| 1.20% of 2-amino-2-methyl-1-propanol | |
| 0.20% of Cremophor ® A 25 | |
| 0.10% of Uvinul ® P 25 | PEG-25 PABA |
| q.s. preservative | |
| q.s. perfume oil | |
| to 100% water | |

### Examples 22 to 28

### Pump Sprays

INCI

| | |
|---|---|
| 4.00% of copolymer from examples 1 to 7 | |
| 1.00% of panthenol | |
| 0.10% of Uvinul ® MS 40 | Benzophenone-4 |
| q.s. preservative | |
| q.s. perfume oil | |
| to 100% water | |

### Examples 29 to 35

### Pump Sprays

INCI

| | |
|---|---|
| 4.00% of copolymer from examples 1 to 7 | |
| 1.00% of panthenol | |
| 0.10% of Uvinul ® M 40 | Benzophenone-3 |
| q.s. preservative | |
| q.s. perfume oil | |
| to 100% ethanol | |

### Examples 36 to 42

### Hair Sprays

INCI

| | |
|---|---|
| 5.00% of copolymer from examples 1 to 7 | |
| 0.10% of silicone oil Dow Corning DC 190 | dimethicone copolyol |
| 35.00% of dimethyl ether | |
| 5.00% of n-pentane | |
| to 100% ethanol | |
| q.s. perfume oil | |

### Examples 43 to 49

### Hair Sprays VOC 55%:

INCI

| | |
|---|---|
| 3.00% of copolymer from examples 1 to 7 | |
| 7.00% of Luviset ® P.U.R. | Polyurethane-1 |
| 40.00% of dimethyl ether | |
| 15.00% of ethanol | |
| q.s. perfume oil | |
| to 100% water | |

### Examples 50 to 56

### Hair Gels

INCI

| | |
|---|---|
| 0.50% of Carbopol ® 980 | Carbomer |
| 3.00% of copolymer from examples 1 to 7 | |
| 0.10% of phythantriol | |
| 0.50% of panthenol | |
| q.s. perfume oil | |
| q.s. preservative | |
| to 100% water | |

### Examples 57 to 64

### Hair Shampoo or Shower Gel

INCI

| | |
|---|---|
| 0.50% of copolymer from examples 1 to 7 | |
| 40.00% of Texapon ® NSO sulfate | sodium laureth |
| 5.00% of Tego Betain ® L 7 | cocamidopropyl betaine |
| 5.00% of Plantacare ® 2000 | decyl glucoside |
| 1.00% of propylene glycol | |
| q.s. citric acid | |
| q.s. preservative | |
| 1.00% of sodium chloride | |
| to 100% water | |

### Use Examples 65 to 71

### Skin Cream

Water/oil cream emulsions were prepared according to the following formulation:

| | Additive | % by wt. |
|---|---|---|
| Cremophor ® A 6 | ceteareth-6 and stearyl alcohol | 2.0 |
| Cremophor ® A 25 | ceteareth-25 | 2.0 |
| Lanette ® O | cetearyl alcohol | 2.0 |
| Imwitor ® 960 K | glyceryl stearate SE | 3.0 |
| Liquid paraffin | | 5.0 |
| Jojoba oil | | 4.0 |
| Luvitol ® EHO | cetearyl octanoate | 3.0 |
| ABIL ® 350 | dimethicone | 1.0 |
| Amerchol ® L 101 | mineral oil and lanolin alcohol | 3.0 |
| Veegum ® Ultra | magnesium aluminum silicate | 0.5 |
| 1,2-Propylene glycol | propylene glycol | 5.0 |
| Abiol | imidazolindinylurea | 0.3 |
| phenoxyethanol | | 0.5 |
| D-Panthenol USP | | 1.0 |
| Polymer (preparation examples 1 to 7) | | 0.5 |
| Water | | to 100 |

### Use Examples 72 to 78

### Shower Gels

Shower gel formulations were prepared according to the following formulation:

| | Additive | % by wt. |
|---|---|---|
| Texapon ® NSO | sodium laureth sulfate | 40.0 |
| Tego ® Betain L7 | cocamidopropyl betaine | 5.0 |
| Plantacare ® 2000 | decyl glucoside | 5.0 |
| Perfume | | 0.2 |
| Polymer according to preparation examples 1 to 7 | | 0.2 |
| Euxyl ® K 100 | benzyl alcohol, | 0.1 |
| | methylchloroiso-thiazolinone, | |
| | methylisothiazolinone | |
| D-Panthenol USP | | 0.5 |
| Citric acid (pH 6-7) | | q.s. |
| NaCl | | 2.0 |
| Water | | to 100 |

### Use Examples

### Moisture Formulations

Formulation A

| | Additive | | % by wt. |
|---|---|---|---|
| a) | Cremophor ® A 6 | ceteareth-6 and | 2.0 |
| | | stearyl alcohol | |
| | Cremophor ® A 25 | ceteareth-25 | 2.0 |
| | Liquid paraffin (high-viscosity) | | 10 |
| | Lannette ® O | cetearyl alcohol | 2.0 |
| | Stearic acid | | 3.0 |
| | Nip-Nip | methylparaben/ | 0.5 |
| | | propylparaben 70:30 | |
| | Abiol | imidazoldinylurea | 0.5 |
| b) | Polymer (preparation examples 1 to 7) | | 3.0 |
| | Water | | to 100.0 |

Both phases were heated to 80° C., phase a) was stirred into b), homogenized and stirred until cold and then brought to pH 6 with 10% strength aqueous NaOH solution.

### Use Examples

### O/W Creams for Skin Moisturizing

| | % by |
|---|---|
| Additive | wt. |
| Glyceryl monostearate | 2.0 |
| Cetyl alcohol | 3.0 |
| Liquid paraffin | 15.0 |
| Vaseline | 3.0 |
| Caprylic/capric triglyceride | 4.0 |
| Octyldodecanol | 2.0 |
| Hydrogenated coconut fat | 2.0 |
| Cetyl phosphate | 0.4 |
| Polymer (preparation examples 1 to 7) | 3.0 |
| Glycerol | 3.0 |
| Sodium hydroxide | q.s. |
| Perfume oil | q.s. |
| Preservative | q.s. |
| Water | to 100 |

### Use Examples

### O/W Lotions

| Additive | % by wt. |
|---|---|
| Stearic acid | 1.5 |
| Sorbitan monostearate | 1.0 |
| Sorbitan monooleate | 1.0 |
| Liquid paraffin | 7.0 |
| Cetyl alcohol | 1.0 |
| Polydimethylsiloxane | 1.5 |
| Glycerol | 3.0 |
| Polymer (preparation examples 1 to 7) | 0.5 |
| Perfume oil | q.s. |
| Preservative | q.s. |
| Water | to 100 |

### Use Examples

### W/O Creams

| | % by |
|---|---|
| Additive | wt. |
| PEG-7 hydrogenated castor oil | 4.0 |
| Wool wax alcohol | 1.5 |
| Beeswax | 3.0 |
| Triglyceride, liquid | 5.0 |
| Vaseline | 9.0 |
| Ozokerite | 4.0 |
| Liquid paraffin | 4.0 |
| Glycerol | 2.0 |
| Polymer (preparation examples 1 to 7) | 2.0 |
| Magnesium sulfate*7H₂0 | 0.7 |
| Perfume oil | q.s. |
| Preservative | q.s. |
| Water | to 100 |

### Use Examples

### Hydrogels for Skin Care

| Additive | % by wt. |
|---|---|
| Polymer (preparation examples 1 to 7) | 3.0 |
| Sorbitol | 2.0 |
| Glycerol | 3.0 |
| Polyethylene glycol 400 | 5.0 |
| Ethanol | 1.0 |
| Perfume oil | q.s. |
| Preservative | q.s. |
| Water | to 100 |

### Use Examples

### Hydrodispersion Gels

| Additive | % by wt. |
|---|---|
| Polymer (preparation examples 1 to 7) | 3.0 |
| Sorbitol | 2.0 |
| Glycerol | 3.0 |
| Polyethylene glycol 400 | 5.0 |
| Triglyceride, liquid | 2.0 |
| Ethanol | 1.0 |
| Perfume oil | q.s. |
| Preservative | q.s. |
| Water | to 100 |

### Use Examples

### Liquid Soaps

| Additive | % by wt. |
|---|---|
| Coconut fatty acid, potassium salt | 15 |
| Potassium oleate | 3 |
| Glycerol | 5 |
| Polymer (preparation examples 1 to 7) | 2 |
| Glycerol stearate | 1 |
| Ethylene glycol distearate | 2 |
| Specific additives, complexing agents, fragrances | q.s. |
| Water | to 100 |

### Use Examples

### Body Care Creams

| Additive | | % by wt. |
|---|---|---|
| Cremophor ® A 6 | ceteareth-6 and stearyl alcohol | 2.0% |
| Cremophor ® A 25 | ceteareth-25 | 2.0% |
| Grape (Vitis Vinifera) seed oil | | 6.0% |
| Glyceryl stearate SE | | 3.0% |
| Cetearyl alcohol | | 2.0% |
| Dimethicone | | 0.5% |
| Luvitol ® EHO | cetearyl octanoate | 8.0% |
| Oxynex ® 2004 | propylene glycol, BHT, ascorbyl | 0.1% |
| | palmitate, glyceryl stearate, | |
| | citric acid | |

| | |
|---|---|
| Preservative | q.s. |
| 1,2-Propylene glycol USP | 3.0% |
| Glycerol | 2.0% |
| EDTA BD | 0.1% |
| D-Panthenol USP | 1.0% |
| Water | to 100 |
| Polymer (preparation examples 1 to 7) | 1.5% |
| Tocopheryl acetate | 0.5% |

In the use examples below, all stated amounts are in % by weight.

### Use Examples

### Liquid Makeups

| A | |
|---|---|
| 1.70 | glyceryl stearate |
| 1.70 | cetyl alcohol |
| 1.70 | ceteareth-6 |
| 1.70 | ceteareth-25 |
| 5.20 | caprylic/capric triglyceride |
| 5.20 | mineral oil |
| B | |
| q.s. | preservative |
| 4.30 | propylene glycol |
| 2.50 | polymer according to preparation examples 1 to 7 |
| 59.50 | distilled water |
| C | |
| q.s. | perfume oil |
| D | |
| 2.00 | iron oxides |
| 12.00 | titanium dioxide |

Preparation:

Phase A and phase B are heated to 80° C. separately from one another. Phase B is then mixed into phase A using a stirrer. Everything is allowed to cool to 40° C. and phase C and phase D are added. Homogenization is repeated.

### Use Examples

### Oil-Free Makeups

| A | |
|---|---|
| 0.35 | veegum |
| 5.00 | butylene glycol |
| 0.15 | xanthan gum |
| B | |
| 53.0 | distilled water |
| q.s. | preservative |
| 0.2 | polysorbate-20 |
| 1.6 | tetrahydroxypropyl ethylenediamine |
| C | |
| 1.0 | silica |
| 2.0 | nylon-12 |
| 4.15 | mica |
| 6.0 | titanium dioxide |
| 1.85 | iron oxides |
| D | |
| 4.0 | stearic acid |
| 1.5 | glyceryl stearate |
| 7.0 | benzyl laurate |
| 5.0 | isoeicosane |
| q.s. | preservative |
| E | |
| 1.0 | distilled water |
| 0.5 | panthenol |
| 0.1 | imidazolidinyl urea |
| 5.0 | polymer according to preparation examples 1 to 7 |

Preparation:

Phase A is wet with butylene glycol, added to phase B and mixed thoroughly. Phase AB is heated to 75° C. Starting materials of phase C are powdered, added to phase AB and thoroughly homogenized. Starting materials of phase D are mixed, heated to 80° C. and added to phase ABC. Mixing is effected for some time until everything is homogeneous. Everything is transferred to a vessel having a propeller mixer. Starting materials of phase E are mixed, added to phase ABCD and thoroughly mixed.

### Use Examples

### Eyeliner

| A | |
|---|---|
| 40.6 | distilled water |
| 0.2 | disodium EDTA |
| q.s. | preservative |
| B | |
| 0.6 | xanthan gum |
| 0.4 | veegum |
| 3.0 | butylene glycol |
| 0.2 | polysorbate-20 |
| C | |
| 15.0 | iron oxide/Al powder/silica (e.g. Sicopearl Fantastico |
| | Gold ™ from BASF) |
| D | |
| 10.0 | distilled water |
| 30.0 | polymer according to preparation examples 1 to 7 |

Preparation:

Phase B is premixed. Phase B is mixed into phase A by means of a propeller mixer, the thickener being allowed to swell. Phase C is wet with phase D, everything is added to phase AB and thorough mixing is effected.

### Use Examples

### Shimmering Gels

| A | |
|---|---|
| 32.6 | distilled water |
| 0.1 | disodium EDTA |
| 25.0 | carbomer (2% strength aqueous solution) |
| 0.3 | preservative |
| B | |
| 0.5 | distilled water |
| 0.5 | triethanolamine |
| C | |
| 10.0 | distilled water |
| 9.0 | polymer according to examples 1 to 7 |
| 1.0 | polyquaternium-46 |
| 5.0 | iron oxide |
| D | |
| 15.0 | distilled water |
| 1.0 | D-panthenol 50 P (panthenol and propylene glycol) |

Preparation:

Using a propeller mixer, the starting materials of phase A are thoroughly mixed in the stated sequence. Phase B is then added to phase A. Slow stirring is effected until everything is homogeneous. Phase C is thoroughly homogenized until the pigments have been well distributed. Phase C and phase D are added to phase AB and thoroughly mixed.

### Use Examples

### Water-Resistant Mascaras

| A | |
|---|---|
| 46.7 | distilled water |
| 3.0 | Lutrol E 400 (PEG-8) |
| 0.5 | xanthan gum |
| q.s. | preservative |
| 0.1 | imidazolidinyl urea |
| 1.3 | tetrahydroxypropyl ethylenediamine |
| B | |
| 8.0 | carnauba wax |
| 4.0 | beeswax |
| 4.0 | isoeicosane |
| 4.0 | polyisobutene |
| 5.0 | stearic acid |
| 1.0 | glyceryl stearate |
| q.s. | preservative |
| 2.0 | benzyl laurate |
| C | |
| 10.0 | iron oxide/Al powder/silica (e.g. Sicopearl Fantastico |
| | Gold ™ from BASF) |
| E | |
| 8.0 | polyurethane-1 |
| 2.0 | polymer according to preparation examples 1 to 7 |

Preparation:

Phase A and phase B are heated to 85° C. separately from one another. The temperature is maintained and phase C is added to phase A and homogenized until the pigments have been uniformly distributed. Phase B is added to phase AC and homogenization is effected for 2-3 minutes. Phase E is then added and slow stirring is effected. Everything is allowed to cool to room temperature.

### Use Examples

### Sunscreen Gels

| Phase A | |
|---|---|
| 1.00 | PEG-40 hydrogenated castor oil |
| 8.00 | octyl methoxycinnamate (Uvinul MC 80 ™ |
| | from BASF) |
| 5.00 | octocrylene (Uvinul N 539 ™ from BASF) |
| 0.80 | octyl triazone (Uvinul T 150 ™ from BASF) |
| 2.00 | butyl methoxydibenzoylmethane (Uvinul BMBM ™ |
| | from BASF) |
| 2.00 | tocopheryl acetate |
| q.s. | perfume oil |
| Phase B | |
| 2.50 | polymer according to preparation examples 1 to 7 |
| 0.30 | acrylates/C10-30 alkyl acrylate crosspolymer |
| 0.20 | carbomer |
| 5.00 | glycerol |
| 0.20 | disodium EDTA |
| q.s. | preservative |
| 72.80 | distilled water |
| Phase C | |
| 0.20 | sodium hydroxide |

Preparation:

The components of phase A are mixed. Phase B is allowed to swell and is stirred into phase A with homogenization. Neutralization is effected with phase C and homogenization is carried out again.

### Use Examples

### Sunscreen Emulsions Containing TiO₂ and ZnO₂

| | |
|---|---|
| Phase A | |
| 6.00 | PEG-7 hydrogenated castor oil |
| 2.00 | PEG-45/dodecyl glycol copolymer |
| 3.00 | isopropyl myristate |
| 8.00 | jojoba (Buxus Chinensis) oil |
| 4.00 | octyl methoxycinnamate (Uvinul MC 80) |
| 2.00 | 4-methylbenzylidene camphor (Uvinul MBC 95) |
| 3.00 | titanium dioxide, dimethicone |
| 1.00 | dimethicone |
| 5.00 | zinc oxide, dimethicone |
| Phase B | |
| 2.00 | polymer according to preparation examples 1 to 7 |
| 0.20 | disodium EDTA |
| 5.00 | glycerol |
| q.s. | preservative |
| 58.80 | distilled water |
| Phase C | |
| q.s. | perfume oil |

Preparation:

The phases A and B are heated separately to about 85° C. Phase B is stirred into phase A and homogenized. Cooling to about 40° C. is effected, phase C is added and homogenization is carried out briefly again.

### Use Examples

### Sun Protection Lotions

| | |
|---|---|
| Phase A | |
| 6.00 | octyl methoxycinnamate (Uvinul MC 80 ™ from BASF) |
| 2.50 | 4-methylbenzylidene camphor (Uvinul MBC 95 ™ |
| | from BASF) |
| 1.00 | octyl triazone (Uvinul T 150 ™ from BASF) |
| 2.00 | butyl methoxydibenzoylmethane (Uvinul BMBM ™ |
| | from BASF) |
| 2.00 | PVP/hexadecene copolymer |
| 5.00 | PPG-3 myristyl ether |
| 0.50 | dimethicone |
| 0.10 | BHT, ascorbyl palmitate, citric acid, |
| | glyceryl stearate, propylene glycol |
| 2.00 | cetyl alcohol |
| 2.00 | potassium cetyl phosphate |
| Phase B | |
| 2.50 | polymer according to preparation examples 1 to 7 |
| 5.00 | propylene glycol |
| 0.20 | disodium EDTA |
| q.s. | preservative |
| 63.92 | distilled water |
| Phase C | |
| 5.00 | mineral oil |
| 0.20 | carbomer |
| Phase D | |
| 0.08 | sodium hydroxide |
| Phase E | |
| q.s. | perfume oil |

Preparation:

The phases A and B are heated separately to about 80° C. Phase B is stirred into phase A with homogenization and further homogenization is carried out briefly. Phase C is converted into suspension, stirred into phase AB, neutralized with phase b and further homogenized. Cooling to about 40° C. is effected, phase E is added and homogenization is effected again.

### Use Examples

### Peelable Face Masks

| Phase A | |
|---|---|
| 57.10 | distilled water |
| 6.00 | polyvinyl alcohol |
| 5.00 | propylene glycol |
| Phase B | |
| 20.00 | alcohol |
| 4.00 | PEG-32 |
| q.s. | perfume oil |
| Phase C | |
| 5.00 | polyquaternium-44 |
| 2.70 | polymer according to preparation examples 1 to 7 |
| 0.20 | allantoin |

Preparation:

Phase A is heated to at least 90° C. and stirred until dissolved. Phase B is dissolved at 50° C. and stirred into phase A. At about 35° C., the ethanol loss is compensated. Phase C is added and mixed in.

### Use Examples

### Face Masks

| Phase A | |
|---|---|
| 3.00 | ceteareth-6 |
| 1.50 | ceteareth-25 |
| 5.00 | cetearyl alcohol |
| 6.00 | cetearyl octanoate |
| 6.00 | mineral oil |
| 0.20 | bisabolol |
| 3.00 | glyceryl stearate |
| Phase B | |
| 2.00 | propylene glycol |
| 5.00 | panthenol |
| 2.80 | polymer according to preparation examples 1 to 7 |
| q.s. | preservative |
| 65.00 | distilled water |
| Phase C | |
| q.s. | perfume oil |
| 0.50 | tocopheryl acetate |

Preparation:

Phases A and B are heated separately to about 80° C. Phase B is stirred into phase A with homogenization and further homogenization is effected briefly. Cooling to about 40° C. is effected, phase C is added and homogenization is carried out again.

### Use Examples

### Body Lotion Foams

| | |
|---|---|
| Phase A | |
| 1.50 | ceteareth-25 |
| 1.50 | ceteareth-6 |
| 4.00 | cetearyl alcohol |
| 10.00 | cetearyl octanoate |
| 1.00 | dimethicone |
| Phase B | |
| 3.00 | polymer according to preparation examples 1 to 7 |
| 2.00 | panthenol |
| 2.50 | propylene glycol |
| q.s. | preservative |
| 74.50 | distilled water |
| Phase C | |
| q.s. | perfume oil |

Preparation:

The phases A and B are heated separately to about 80° C. Phase B is stirred into phase A and homogenized. Cooling to about 40° C. is effected, phase C is added and homogenization is carried out again briefly. Filling: 90% of active ingredient and 10% of propane/butane at 3.5 bar (20° C.).

### Use Examples

### Face Lotion for Dry and Sensitive Skin

| Phase A | |
|---|---|
| 2.50 | PEG-40 hydrogenated castor oil |
| q.s. | perfume oil |
| 0.40 | bisabolol |
| Phase B | |
| 3.00 | glycerol |
| 1.00 | hydroxyethyl cetyldimonium phosphate |
| 5.00 | witch hazel (Hamamelis Virginiana) distillate |
| 0.50 | panthenol |
| 0.50 | polymer according to preparation examples 1 to 7 |
| q.s. | preservative |
| 87.60 | distilled water |

Preparation:

Phase A is dissolved to give a clear solution. Phase B is stirred into phase A.

### Use Examples

### Face Wash Pastes with Peeling Effect

| Phase A | |
|---|---|
| 70.00 | distilled water |
| 3.00 | polymer according to preparation examples 1 to 7 |
| 1.50 | carbomer |
| q.s. | preservative |
| Phase B | |
| q.s. | perfume oil |
| 7.00 | potassium cocoyl hydrolyzed protein |
| 4.00 | cocamidopropyl betaine |
| Phase C | |
| 1.50 | triethanolamine |
| Phase D | |
| 13.00 | polyethylene (Luwax A ™ from BASF) |

Preparation:

Phase A is allowed to swell. Phase B is dissolved to give a clear solution. Phase B is stirred into phase A. Neutralization is effected with phase C. Phase D is then stirred in.

### Use Examples

### Face Soaps

| | |
|---|---|
| Phase A | |
| 25.0 | potassium cocoate |
| 20.0 | disodium cocoamphodiacetate |
| 2.0 | lauramide DEA |
| 1.0 | glycol stearate |
| 2.0 | polymer according to preparation examples 1 to 7 |
| 50.0 | distilled water |
| q.s. | citric acid |
| Phase B | |
| q.s. | preservative |
| q.s. | perfume oil |

Preparation:

Phase A is heated to 70° C. with stirring until everything is homogeneous. The pH is brought to 7.0 to 7.5 with citric acid. Everything is allowed to cool to 50° C. and phase B is added.

### Use Examples

### Face Cleaning Milk Type O/W

| Phase A | |
|---|---|
| 1.50 | ceteareth-6 |
| 1.50 | ceteareth-25 |
| 2.00 | glyceryl stearate |
| 2.00 | cetyl alcohol |
| 10.00 | mineral oil |
| Phase B | |
| 5.00 | propylene glycol |
| q.s. | preservative |
| 1.0 | polymer according to preparation examples 1 to 7 |
| 66.30 | distilled water |
| Phase C | |
| 0.20 | carbomer |
| 10.00 | cetearyl octanoate |
| Phase D | |
| 0.40 | tetrahydroxypropyl ethylenediamine |
| Phase E | |
| q.s. | perfume oil |
| 0.10 | bisabolol |

Preparation:

The phases A and B are heated separately to about 80C. Phase B is stirred into phase A with homogenization and homogenization is carried out again briefly. Phase C is converted into a suspension and stirred into phase AB, neutralization is effected with phase D and homogenization is carried out again. Cooling to about 40° C. is effected, phase E is added and homogenization is carried out again.

### Use Examples

### Transparent Soaps

| | |
|---|---|
| 4.20 | sodium hydroxide |
| 3.60 | distilled water |
| 2.0 | polymer according to preparation examples 1 to 7 |
| 22.60 | propylene glycol |
| 18.70 | glycerol |
| 5.20 | cocoamide DEA |
| 10.40 | cocamine oxide |
| 4.20 | sodium lauryl sulfate |
| 7.30 | myristic acid |
| 16.60 | stearic acid |
| 5.20 | tocopherol |

Preparation:

All ingredients are mixed. The mixture is melted at 85° C. to give a clear melt. It is immediately poured into the mold.

### Use Examples

### Peeling Creams, Type O/W

| Phase A | |
|---|---|
| 3.00 | ceteareth-6 |
| 1.50 | ceteareth-25 |
| 3.00 | glyceryl stearate |
| 5.00 | cetearyl alcohol, sodium cetearyl sulfate |
| 6.00 | cetearyl octanoate |
| 6.00 | mineral oil |
| 0.20 | bisabolol |
| Phase B | |
| 2.00 | propylene glycol |
| 0.10 | disodium EDTA |
| 3.00 | polymer according to preparation examples 1 to 7 |
| q.s. | preservative |
| 59.70 | distilled water |
| Phase C | |
| 0.50 | tocopheryl acetate |
| q.s. | perfume oil |
| Phase D | |
| 10.00 | polyethylene |

Preparation:

The phases A and B are heated separately to about 80° C. Phase B is stirred into phase A and homogenized. Cooling to about 40° C. is effected, phase C is added and homogenization is carried out again briefly. Phase D is then stirred in.

### Use Examples

### Shaving Foams

| | |
|---|---|
| 6.00 | ceteareth-25 |
| 5.00 | poloxamer 407 |
| 52.00 | distilled water |
| 1.00 | triethanolamine |
| 5.00 | propylene glycol |
| 1.00 | PEG-75 lanolin oil |
| 5.00 | polymer according to preparation examples 1 to 7 |
| q.s. | preservative |
| q.s. | perfume oil |
| 25.00 | sodium laureth sulfate |

Preparation:

Everything is weighed together and then stirred until dissolved. Filling: 90 parts of active substance and 10 parts of propane/butane mixture 25:75.

### Use Examples

### After Shave Balsam

| Phase A | |
|---|---|
| 0.25 | acrylates/C10-30 alkyl acrylate crosspolymer |
| 1.50 | tocopheryl acetate |
| 0.20 | bisabolol |
| 10.00 | caprylic/capric triglyceride |
| q.s. | perfume oil |
| 1.00 | PEG-40 hydrogenated castor oil |
| Phase B | |
| 1.00 | panthenol |
| 15.00 | alcohol |
| 5.00 | glycerol |
| 0.05 | hydroxyethyl cellulose |
| 1.92 | polymer according to preparation examples 1 to 7 |
| 64.00 | distilled water |
| Phase C | |
| 0.08 | sodium hydroxide |

Preparation:

The components of phase A are mixed. Phase B is stirred into phase A with homogenization, and homogenization is carried out again briefly. Neutralization is effected with phase C and homogenization is carried out again.

### Use Examples

### Personal Hygiene Creams

| | |
|---|---|
| Phase A | |
| 2.00 | ceteareth-6 |
| 2.00 | ceteareth-25 |
| 2.00 | cetearyl alcohol |
| 3.00 | glyceryl stearate SE |
| 5.00 | mineral oil |
| 4.00 | jojoba (Buxus Chinensis) oil |
| 3.00 | cetearyl octanoate |
| 1.00 | dimethicone |
| 3.00 | mineral oil, lanolin alcohol |
| Phase B | |
| 5.00 | propylene glycol |
| 0.50 | veegum |
| 1.00 | panthenol |
| 1.70 | polymer according to preparation examples 1 to 7 |
| 6.00 | polyquaternium-44 |
| q.s. | preservative |
| 60.80 | distilled water |
| Phase C | |
| q.s. | perfume oil |

Preparation:

The phases A and B are heated separately to about 80C. Phase B is homogenized.

Phase B is stirred into phase A with homogenization and homogenization is carried out briefly again. Cooling to about 40° C. is effected, phase C is added and homogenization is carried out briefly again.

### Use Examples

### Toothpaste

| Phase A | |
|---|---|
| 34.79 | distilled water |
| 3.00 | polymer according to preparation examples 1 to 7 |
| 0.30 | preservative |
| 20.00 | glycerol |
| 0.76 | sodium monofluorophosphate |
| Phase B | |
| 1.20 | sodium carboxymethylcellulose |
| Phase C | |
| 0.80 | aroma oil |
| 0.06 | saccharin |
| 0.10 | preservative |
| 0.05 | bisabolol |
| 1.00 | panthenol |
| 0.50 | tocopheryl acetate |
| 2.80 | silica |
| 1.00 | sodium lauryl sulfate |
| 7.90 | dicalcium phosphate anhydrate |
| 25.29 | dicalcium phosphate dihydrate |
| 0.45 | titanium dioxide |

Preparation:

Phase A is dissolved. Phase B is sprinkled into phase A and dissolved. Phase C is added and stirring is carried out under reduced pressure at room temperature for about 45 minutes.

### Use Examples

### Mouthwash

| Phase A | |
|---|---|
| 2.00 | aroma oil |
| 4.00 | PEG-40 hydrogenated castor oil |
| 1.00 | bisabolol |
| 30.00 | alcohol |
| Phase B | |
| 0.20 | saccharin |
| 5.00 | glycerol |
| q.s. | preservative |
| 5.00 | poloxamer 407 |
| 0.5 | polymer according to preparation examples 1 to 7 |
| 52.30 | distilled water |

Preparation:

Phase A and phase B are dissolved separately to give clear solutions. Phase B is stirred into phase A.

### Use Examples

### Prosthesis Adhesive

| Phase A | |
|---|---|
| 0.20 | bisabolol |
| 1.00 | beta-carotene |
| q.s. | aroma oil |
| 20.00 | cetearyl octanoate |
| 5.00 | silica |
| 33.80 | mineral oil |
| Phase B | |
| 5.00 | polymer according to preparation examples 1 to 7 |
| 35.00 | PVP (20% strength solution in water) |

Preparation:

Thoroughly mix phase A and stir phase B into phase A.

### Use Example 32

### Skin Care Cream, type O/W

| | |
|---|---|
| Phase A | |
| 8.00 | cetearyl alcohol |
| 2.00 | ceteareth-6 |
| 2.00 | ceteareth-25 |
| 10.00 | mineral oil |
| 5.00 | cetearyl octanoate |
| 5.00 | dimethicone |
| Phase B | |
| 3.00 | polymer according to preparation examples 1 to 7 |
| 2.00 | panthenol, propylene glycol |
| q.s. | preservative |
| 63.00 | distilled water |
| Phase C | |
| q.s. | perfume oil |

Preparation:

Phases A and B are heated separately to about 80° C. Phase B is stirred into phase A with homogenization, and further homogenization is effected. Cooling to about 40° C. is effected, phase C is added and homogenization is carried out again.

### Use Examples

### Skin Care Creams, Type W/O

| | |
|---|---|
| Phase A | |
| 6.00 | PEG-7 hydrogenated castor oil |
| 8.00 | cetearyl octanoate |
| 5.00 | isopropyl myristate |
| 15.00 | mineral oil |
| 2.00 | PEG-45/dodecyl glycol copolymer |
| 0.50 | magnesium stearate |
| 0.50 | aluminum stearate |
| Phase B | |
| 3.00 | glycerol |
| 3.30 | polymer according to preparation examples 1 to 7 |
| 0.70 | magnesium sulfate |
| 2.00 | panthenol |
| q.s. | preservative |
| 48.00 | distilled water |
| Phase C | |
| 1.00 | tocopherol |
| 5.00 | tocopheryl acetate |
| q.s. | perfume oil |

Preparation:

The phases A and B are heated separately to about 80° C. Phase B is stirred into phase A and homogenized. Cooling to about 40° C. is effected, phase C is added and homogenization is carried out again briefly.

### Use Examples

### Lip Care Creams

| | |
|---|---|
| Phase A | |
| 10.00 | cetearyl octanoate |
| 5.00 | polybutene |
| Phase B | |
| 0.10 | carbomer |
| Phase C | |
| 2.00 | ceteareth-6 |
| 2.00 | ceteareth-25 |
| 2.00 | glyceryl stearate |
| 2.00 | cetyl alcohol |
| 1.00 | dimethicone |
| 1.00 | benzophenone-3 |
| 0.20 | bisabolol |
| 6.00 | mineral oil |
| Phase D | |
| 8.00 | polymer according to preparation examples 1 to 7 |
| 3.00 | panthenol |
| 3.00 | propylene glycol |
| q.s. | preservative |
| 54.00 | distilled water |
| Phase E | |
| 0.10 | triethanolamine |
| Phase F | |
| 0.50 | tocopheryl acetate |
| 0.10 | tocopherol |
| q.s. | perfume oil |

Preparation:

Phase A is dissolved to give a clear solution. Phase B is added and homogenized. Phase C is added and-melted at 80° C. Phase D is heated to 80° C. Phase D is added to phase ABC and homogenized. Cooling to about 40° C. is effected, phase E and phase F are added and homogenization is carried out again.

### Use Examples

### Glossy Lipsticks

| Phase A | |
|---|---|
| 5.30 | candelilla (Euphorbia Cerifera) wax |
| 1.10 | beeswax |
| 1.10 | microcrystalline wax |
| 2.00 | cetyl palmitate |
| 3.30 | mineral oil |
| 2.40 | castor oil, glyceryl ricinoleate, octyldodecanol, |
| | carnauba, candelilla wax, |
| 0.40 | bisabolol |
| 16.00 | cetearyl octanoate |
| 2.00 | hydrogenated cocoglycerides |
| q.s | preservative |
| 1.00 | polymer according to preparation examples 1 to 7 |
| 60.10 | castor (Ricinus Communis) oil |
| 0.50 | tocopheryl acetate |
| Phase B | |
| 0.80 | C. I. 14 720:1, Acid Red 14 Aluminum Lake |
| Phase C | |
| 4.00 | mica, titanium dioxide |

Preparation:

The components of phase A are weighed in and melted. Phase B is incorporated homogeneously. Phase C is added and stirred in. Cooling to room temperature is effected with stirring.

1. A graft copolymer which is obtainable by free radical graft copolymerization of
a) N-vinylformamide,
b) one or more copolymerizable monomers of the formula (I) 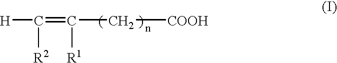
where n is 0 or 1 and R¹ and R², independently of one another, are H, C₁-C₄-alkyl, CN or COOH,
and/or one or more unsaturated sulfonic acids
and/or one or more unsaturated phosphonic acids onto a polymeric grafting base c) of the formula

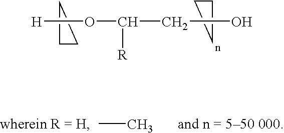

2. A graft polymer according to claim 1, wherein the free radical graft copolymerization is carried out in the presence of a crosslinking agent d).

3. A graft polymer according to claim 1, wherein the monomer b) is a compound of the formula (I).

4. A graft polymer according to claim 1, wherein a polymeric grafting base c) having a molecular weight of from 500 to 50 000, is used.

5. A method of preparing a cosmetic composition comprising adding of a graft polymer according to claim 1 to a cosmetic formulation.

6. A method as claimed in wherein said graft polymer is added as a thickener to said cosmetic formulation.

7. A graft polymer according to claim 1, wherein a polymeric grafting base c) having a molecular weight of from 5000 to 40,000, is used.

8. A method as claimed in claim 5 wherein said cosmetic composition is a hair cosmetic.
